(19) United States
(12) Patent Application Publication
(10) Pub. No.: US 2018/0110925 A1
(43) Pub. Date: Apr. 26, 2018

(54) DRUG DELIVERY

(71) Applicant: Summit Street Medical LLC, Wallingford, CT (US)

(72) Inventors: Robert F. Rioux, Ashland, MA (US); Matthew Laplaca, Franklin, MA (US); Brian Grasso, Wallingford, CT (US); Matt Bomes, Wellesley, MA (US); George Bourne, Boston, MA (US)

(21) Appl. No.: 15/784,939

(22) Filed: Oct. 16, 2017

Related U.S. Application Data
(60) Provisional application No. 62/411,310, filed on Oct. 21, 2016.

(51) Int. Cl. A61M 5/20; A61M 5/315

(52) U.S. Cl. CPC A61M 5/2033; A61M 5/31578; A61M 2005/206; A61M 2202/04; A61M 2209/088; A61M 5/2053

(57) ABSTRACT

A wearable drug delivery device has a drug delivery pack housing a syringe with a drug dose and a separate power pack for providing the energy needed to auto-inject the dose into the user. A flexible conductor or rigid connector couples the two packs together and transits the energy released from the power pack to the drug delivery pack.

### CROSS REFERENCE TO RELATED APPLICATIONS

This application claims the benefit under 35 U.S.C. § 119(e) of U.S. Provisional Application Ser. No. 62/411,310 filed on Oct. 21, 2016 the entire disclosure of which is incorporated herein by reference.

### TECHNICAL FIELD

The present invention relates to autoinjectors and in particular to an autoinjector having separate parts allowing it to be worn by a user.

### BACKGROUND

Ingesting, inhaling, and/or injecting certain allergens, toxins, and/or other substances can cause profound reactions for at least some and/or all people and/or animals. For example, certain people are highly allergic to certain substances, such as peanuts, shellfish, particular drugs, certain proteins, bee venom, insect bites, etc. The allergic response can lead to anaphylactic shock, which can cause a sharp drop in blood pressure, hives, and/or substantial breathing difficulties caused by severe airway constriction. As another example, inhalation of certain nerve agents can cause severe physiological trauma. Responding rapidly to such exposures can prevent injury and/or death. For example, in response to an exposure leading to anaphylactic shock, an injection of epinephrine (i.e., adrenaline) can provide substantial and/or complete relief from the reaction. As another example, injection of an antidote to a nerve agent can greatly reduce and/or eliminate the potential harm of the exposure. As yet another example, rapid injection of certain drugs, such as a beta blocker, blood thinner, nitroglycerine, antihistamines, insulin, and opioids, etc., can provide substantial relief from various dangerous medical conditions.

An autoinjector is a medical device designed to deliver one or more doses of a particular drug in a manner that facilitates self-administration of the drug via a syringe. By design, autoinjectors are easy to use and are intended to be used by patients or by untrained personnel. They typically are self-contained and designed to require only a few basic steps to operate.

### SUMMARY

It is a challenge to package components into a form factor that allows a user to wear a medical device. The medical device can include a syringe, a drug dose, and a source of stored energy needed to auto-inject the dose into the user. A solution to the challenge is a wearable drug delivery device having two separate parts, cases or housings. One part of the device can house the syringe and the drug dose, and this first part can be called the drug delivery pack. The second part of the device can house the stored energy source, and this second part can be called the power pack. The two packs can be coupled together in such a way that the energy released from the stored energy source in the power pack is conveyed to the drug delivery pack and used to move the drug dose through the syringe and into the user via the syringe.

An exemplary wearable drug delivery device includes a stored energy source disposed within a first housing. This device further includes a second housing comprising a syringe movable within the second housing between a withdrawn position and an extended position. The syringe includes a needle and a drug container in fluid communication with each other. The drug container can be filled with a dose of epinephrine or insulin. This device further includes a flexible conductor connected between the first housing and the second housing. The flexible conductor is configured to convey energy released from the stored energy source which moves the syringe to the extended position and extends the needle beyond the second housing.

The stored energy source can be a spring and the flexible conductor can be a Bowden cable.

The stored energy source can be a compressed gas and the flexible conductor can be a lumen.

The wearable drug delivery device can further include a release mechanism coupled to the stored energy source and configured to discharge stored energy. A trigger mechanism can be disposed at one end of the second housing and coupled to the release mechanism such that, in response to the trigger mechanism being activated, the release mechanism discharges the stored energy.

The wearable drug delivery device can further includes a release mechanism coupled to the stored energy source and configured to release stored energy. A trigger mechanism can be disposed at one end of the first housing and coupled to the release mechanism such that in response to the trigger mechanism being activated, the release mechanism discharges the stored energy.

The first housing can further include a first concave surface and the second housing can further include a second concave surface opposite the first concave surface. Each of the first and second concave surfaces extends along a longitudinal axis lying between the first housing and the second housing. Each of the first and second concave surfaces has a concavity defined by a point along the longitudinal axis. The first and second concave surfaces can be configured to conform to the human wrist.

The wearable drug delivery device can further include a cover hingedly attached to the first housing and operable between an initial close position and final open position. An interlock can be in releaseable engagement with the cover. When the interlock in unlocked, the cover swings from initial close position to the final open position. The interlock can include a recess defined by the first housing and a projection extending from the second housing. The interlock unlocks when the projection is inserted into the recess.

The second housing can include an open end through which the needle extends when the syringe is in the extended position. The wearable drug delivery device can further include a seal at the open end for keeping the needle sterile when the syringe is in the withdrawn position.

Another exemplary wearable drug delivery device includes a first housing including a first longitudinal axis and a first concave surface extending in the direction of the first longitudinal axis. The concavity of the first concave surface is defined by a point along an axis offset and parallel to the first longitudinal axis. The first housing further includes a stored energy source is disposed within the first housing. This device further includes a first connector disposed at an end of the first housing. The device further includes a second housing having a second longitudinal axis. The second housing includes a second concave surface extending in the direction of the second longitudinal axis. The concavity of the second concave surface is defined by a second point along an axis offset and parallel to the second longitudinal axis. The second housing includes a syringe that is movable within the second housing between a withdrawn position and an extended position. The syringe includes a needle and a drug container in fluid communication with each other. The drug container can be filled with a dose of epinephrine or insulin. The device further includes a second connector disposed at an end of the second housing. The second connector is adapted to couple the first connector of the first housing. The device further includes a band releasably coupled to the first housing and the second housing. When coupled to the band, the first concave surface of the first housing and the second concave surface of the second housing are opposite each other.

The stored energy source can be any one of a pre-compressed spring or compressed gas.

The first connector and the second connecting can be coaxially aligned with the first longitudinal axis and the second longitudinal axis, respectively. The first connector can be a recess defined in the end of the first housing and the second connector can be a protrusion extending from the end of the second housing. The recess can include radial slots and the protrusion can include radial tabs corresponding to the radial slots.

### BRIEF DESCRIPTION OF THE DRAWINGS

The foregoing and other objects, features and advantages will be apparent from the following more particular description of the embodiments, as illustrated in the accompanying drawings in which like reference characters refer to the same parts throughout the different views. The drawings are not necessarily to scale, emphasis instead being placed upon illustrating the principles of the embodiments.

FIG. 1 is a perspective view of a first example of the wearable drug delivery device with a power pack connected to a drug delivery pack by a flexible conductor.

FIG. 2 is a side cut-away view of the first example of the wearable drug delivery device.

FIG. 3 is a top cut-away view of the first example of the wearable drug delivery device.

FIG. 4 is a perspective view of a second example of the wearable drug delivery device with a power pack connected to a drug delivery pack by a flexible lumen.

FIG. 5 is a side cut-away view of the second example of the wearable drug delivery device.

FIGS. 6A and 6B are views of the second example of the wearable drug delivery device with an interlock and a cover for preventing the wearable drug delivery device from being triggered, accidentally.

FIG. 7 is a perspective view of a third example of the wearable drug delivery device being worn around a user's wrist with a power pack, a drug delivery pack, and a rigid connection for joining the power pack to the drug delivery pack.

FIG. 8 is another perspective view of the third example of the wearable drug delivery.

FIGS. 9A and 9B are views of the third example of the wearable drug delivery with the power pack and drug delivery pack separated and connected, respectively.

FIG. 10 is a side cut-away view of the third example of the wearable drug delivery with the power pack and drug delivery pack separated.

### DETAILED DESCRIPTION

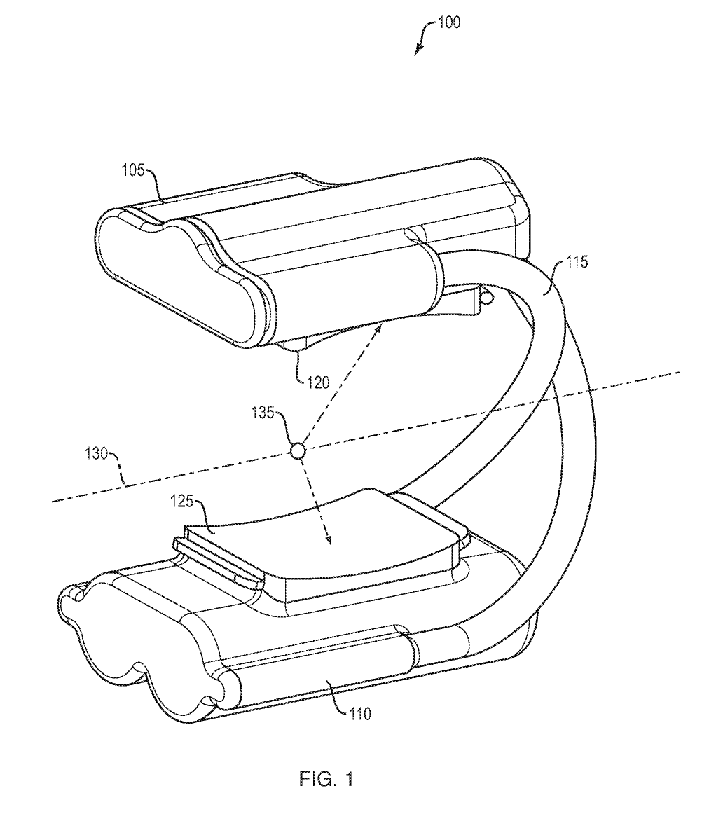

FIG. 1 shows an example wearable drug delivery 100 having a drug delivery pack 105, a power pack 110, and a flexible conductor 115 (two shown but there may be more or fewer). The flexible conductor 115 connects the drug delivery pack 105 to the power pack 110 so that the wearable drug delivery device 100 can be wrapped around a user's wrist or arm, for example. The drug delivery pack 105 includes a first concave surface 120 and the power pack 110 includes a second concave surface 125 opposing the first concave surface 120 conforming to the round shape of the user's wrist, for example. FIG. 1 shows a longitudinal axis 130 of the wearable drug delivery device 100. The first concave surface 120 and the second concave surface 125 extend in the direction of the longitudinal axis 130 and have concavity defined by a point 135 along the longitudinal axis 130.

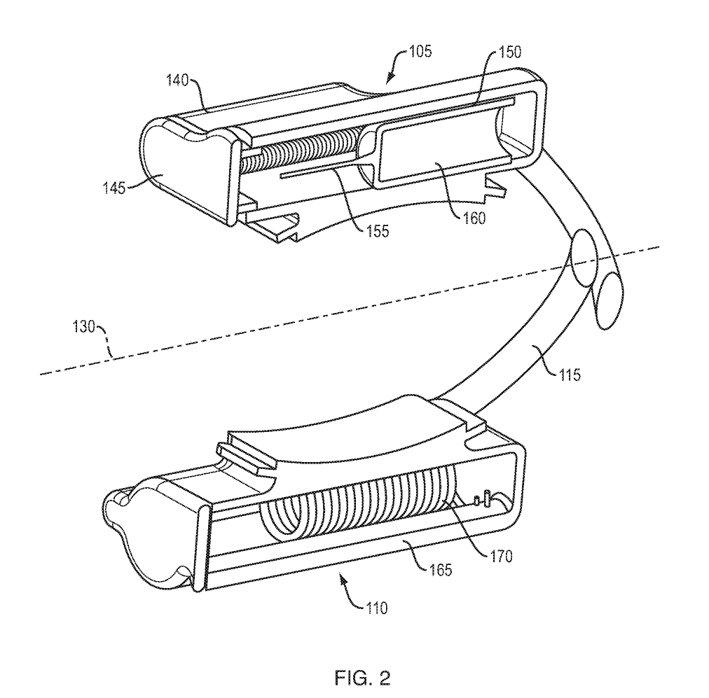

FIG. 2 shows an example of the drug delivery pack 105 having a housing 140 and a trigger portion 145. The housing 140 contains a syringe 150 consisting of a needle 155 at the forward end attached to a drug container 160 that is fitted with a sliding plunger (not shown) at the rear end. By way of non-limiting example, the drug container 160 can be filled with a dose of epinephrine or insulin. The syringe 160 is movable within the housing 140 in the direction of the longitudinal axis 130 from a proximal position (shown) to a distal position. For ease of reference, the proximal position within the housing 140 is called the “withdrawn position” and the distal position within the housing 140 is called the “extended position.” Additionally, the proximal-to-distal direction is referred to as the “downward direction,” and the opposite direction is the “upward direction.”

Moving the trigger portion 145 towards the housing 140 (e.g., pressing the trigger portion 145 against the user's thigh) causes the syringe 150 to move downwardly with the needle 155 extending beyond the trigger portion 145. In a convenient example of the wearable drug delivery device 100, the drug delivery pack 105 is sealed to maintain sterility. In the example, the needle 155 pierces through a sterile seal. In other examples, the drug delivery pack 105 further includes a trigger guard (not shown) separating the housing 140 from the trigger portion 145. The trigger guard prevents the drug delivery pack 105 from being accidently triggered.

FIG. 2 further shows an example of the power pack 110 having a housing 165 and a spring 170 contained within the housing 165. The spring 170 stores mechanical energy used to move the syringe 150 downwardly, to the insert the needle 155 into the user, and to inject the drug dose. The spring 170 includes a fixed end, which is attached to the housing 165 and cannot move, and a movable end. The movable end can move relative to the housing 165 and is coupled to the flexible conductor 115. A release mechanism (not shown) engages the movable end and stops the movable end from moving. The release mechanism is coupled to the trigger portion 145 (described above) by way of the flexible conductor 115 or a separate linkage. Moving the trigger portion 145 towards the housing 140 activates the release mechanism. The release mechanism, in turn, releases the movable end of the spring 170, thereby releasing the mechanical energy stored in the spring 170.

In one example, the spring 170 is a compression spring, i.e., the spring 170 operates under a compression load and its length shortens when the compression load is applied. The release mechanism maintains a compression load holding the spring 170 under compression. Activating the release mechanism removes the compression load from the spring 170 and releases the mechanical energy stored in the spring 170. In turn, the movable end moves in a direction away from the fixed end of the spring 170, which for ease of reference is called a “push.” The flexible conductor 115 then conveys the released mechanical energy to the drug delivery pack 105, as will be described in greater detail below.

In another example, the spring 170 is a tension spring, i.e., the spring 170 operates under a tension load and its length shortens when the tension load is applied. The release mechanism maintains a tension load holding the spring 170 under tension. Activating the release mechanism removes the tension load from the spring 170 and releases the mechanical energy stored in the compression spring. In turn, the movable end moves in a direction toward the fixed end of the spring 170, which for ease of reference is called a “pull.” The flexible conductor 115 then conveys the released mechanical energy to the drug delivery pack 105.

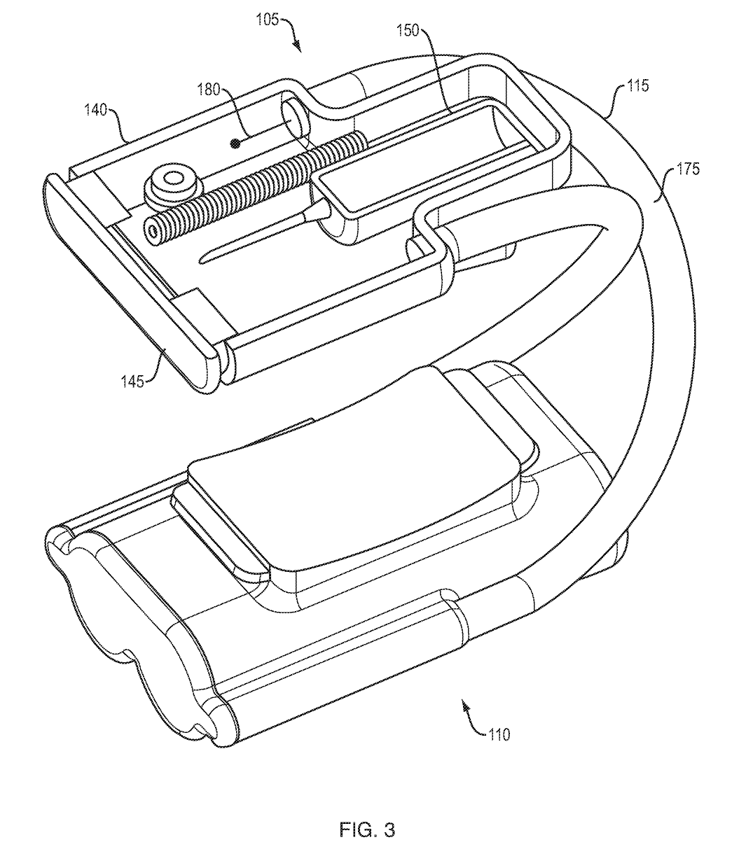

FIG. 3 shows an example of the flexible conductor 115 including a hollow outer cable housing 175 and inner cable 180 movable within the hollow outer cable housing 175. The flexible conductor 115 conveys mechanical energy from the power pack 110 to the drug delivery pack 105 by way of movement of the inner cable 180 relative to the hollow outer cable housing 175. For example, the spring 170 pushes the inner cable 180 relative the hollow outer cable 175. The inner cable 180 in turn pushes the syringe 150, moving the syringe 150 from the withdrawn position to the extended position.

The hollow outer cable housing 175 is generally of composite construction, consisting of an inner lining, a longitudinally incompressible layer, such as a helical winding or a sheaf of steel wire, and a protective outer covering. The inner cable 180 may a solid wire or a wire rope made up of smaller individual strands. The flexible conductor 115 may be constructed to hold a shape that facilitates wearing the wearable drug delivery device 100 on the user's wrist or arm. For example, the flexible conductor 115 includes members, such as wire form springs, giving the flexible conductor 115 a desired shape and flexibility. In another example, the flexible conductor 115 is made from an elastomer that may or may not be reinforced with braids or coils. One example of the flexible conductor 115 is a Bowden cable.

The flexible conductor 115 can convey/transfer the energy released from the spring 170 to the syringe 150 in a number of different ways involving additional components. Example components include a bell crank, a lever, or other part(s) for changing the direction of the conveyed energy. Other examples include a rack and pinion, a gear, a linkage, etc.

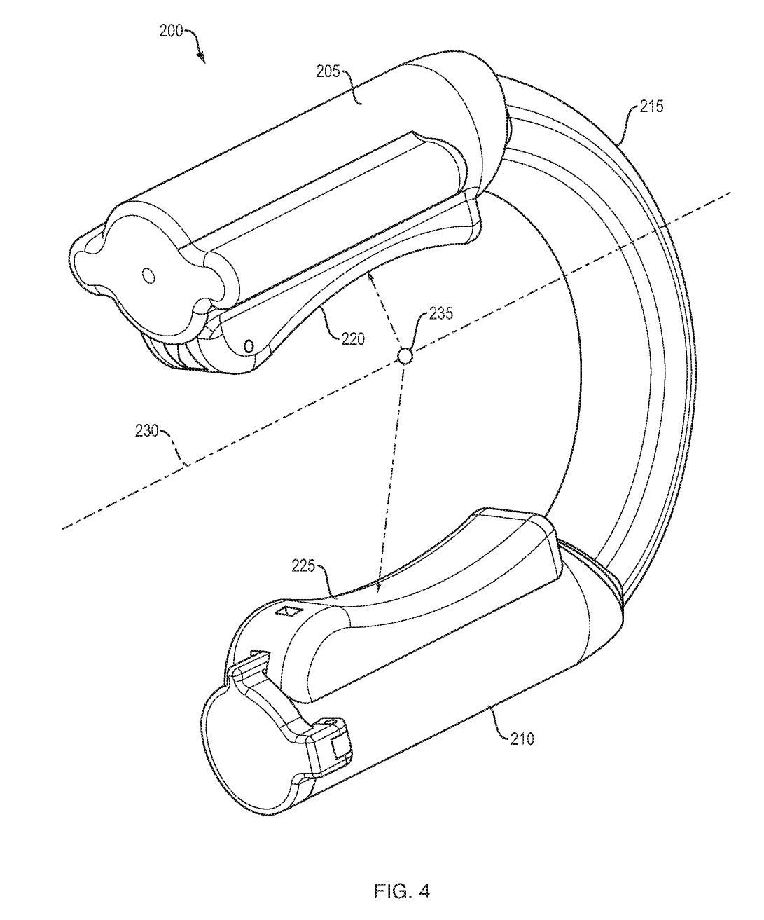

FIG. 4 shows an example wearable drug delivery 200 having a drug delivery pack 205, a power pack 210, and a flexible lumen 215. The flexible lumen 215 connects to the drug delivery pack 205 to the power pack 210 so that the wearable drug delivery device 200 can be wrapped around a user's wrist or arm, for example. The drug delivery pack 205 includes a first concave surface 220 and the power pack 210 includes a second concave surface 225 opposing the first concave surface 220 conforming to the round shape of the user's wrist, for example. FIG. 4 shows a longitudinal axis 230 of the wearable drug delivery device 200. The first concave surface 220 and the second concave surface 225 extend in the direction of the longitudinal axis and have concavity defined by a point 235 along the longitudinal axis 230.

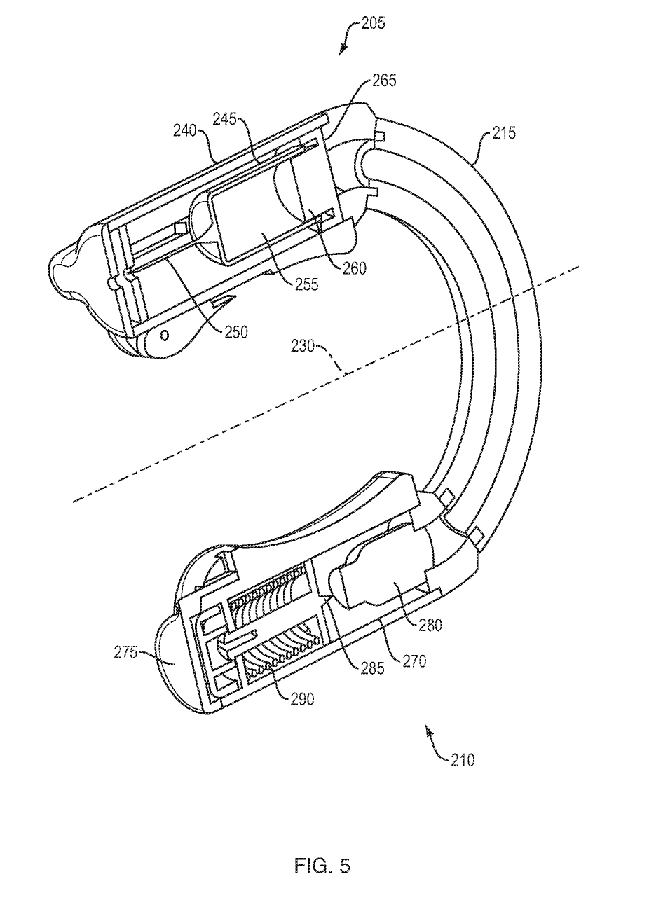

FIG. 5 shows an example of the drug delivery pack 205 having a housing 240. The housing 240 contains a syringe 245 consisting of a needle 250 at the forward end attached to a drug container 255 that is fitted with a sliding plunger 260 at the rear end. By way of non-limiting example, the drug container 255 can be filled with a dose of epinephrine or insulin. Moving the plunger 260 from a proximal position (shown) within the housing 240 to a distal position forces a drug dose out of the drug container 255. An actuator 265 moves the syringe 245 within the housing 240 in the direction of the longitudinal axis 230 from a proximal position (shown) to a distal position. For ease of reference, the proximal position within the housing 240 is called the “withdrawn position” and the distal position within the housing 240 is called the “extended position.” Additionally, the proximal-to-distal direction is referred to as the “downward direction,” and the opposite direction is the “upward direction.”

FIG. 5 further shows an example of the power pack 210 having a housing 270 and a trigger portion 275. The housing 270 holds a container of compressed gas 280 (e.g., CO₂) used to move the syringe 245 downwardly, to the insert the needle 250 into the user, and to inject the drug dose. The housing 270 further holds a puncturer 285 facing the compressed gas container 280. The puncturer 285 is attached to a pre-compressed spring 290 that is held under compression by a release mechanism (not shown). The release mechanism in turn is coupled to the trigger portion 275 by a linkage or other similar type of connection and is activated by moving the trigger portion 275 towards the housing 270. When activated, the release mechanism releases the pre-compressed spring 290 causing the puncturer 285 to move towards the compressed gas container 280 and puncture it.

Upon puncturing the compressed gas container 280, compressed gas escapes and flows from the power pack 210 to the drug delivery pack 205 through the flexible lumen 215. At the drug delivery pack 205, the flowing gas meets the actuator 265 and applies pressure causing the actuator 265 to move in the downward direction (i.e., from proximal to distal). In turn, the actuator 265 moves the syringe 245 in the downward direction causing the needle 250 to exit the drug delivery pack 205. The syringe 245 continues to move until a stop within the drug delivery pack housing 240 prevents further movement. At this point, the needle 250 extends a certain distance beyond the drug delivery pack housing 240. Additional downward movement by the actuator 265 causes the plunger 260 to move downwardly within the drug container 255, thereby expelling the drug dose from the drug container, through the needle 250, and into the user.

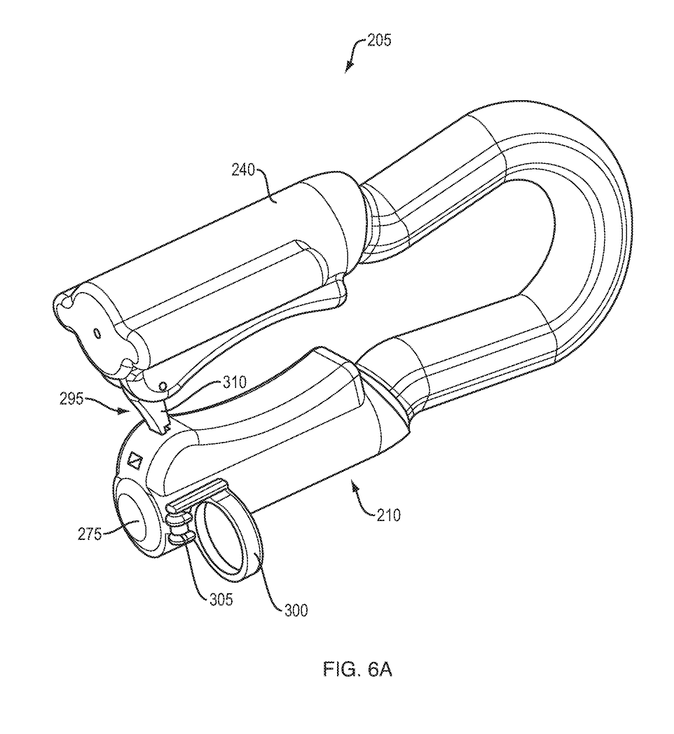

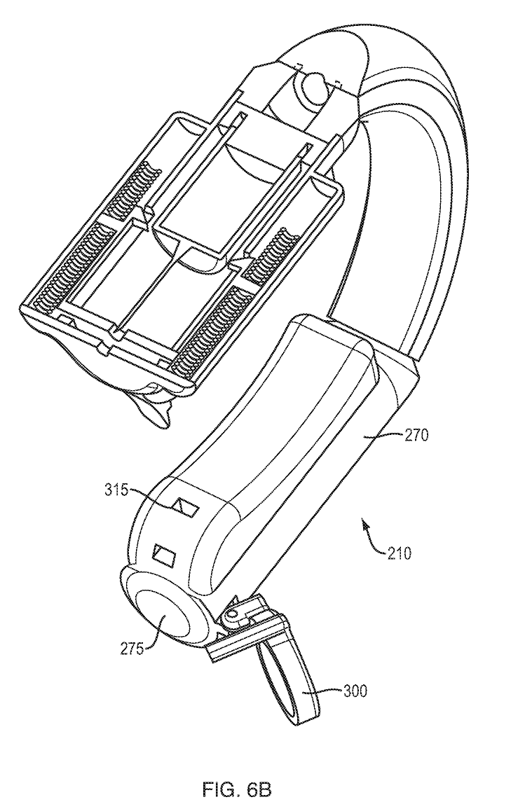

FIGS. 6A and 6B show an interlock 295 and a cover 300 for preventing the wearable drug delivery device 200 from being triggered, accidentally. In the example shown, the cover 300 is attached to the power pack 210 by a hinge 305. The cover 300 operates between a close position that shields the trigger portion 275, and an open position that exposes the trigger portion 275 (shown). When the wearable drug delivery device 200 is being worn, the cover 300 is in the close position. When the wearable drug device 200 is being used to auto-inject a drug dose, the cover 300 is in the open position. Opening the cover 300 to expose the trigger portion 275 requires first the unlocking the interlock 295.

The interlock 295 includes a first part on the drug delivery pack 205 and a second part on the power pack 210 that are combined in order to unlock the interlock 295. In the example shown, the first part is a tab 310 extending from the drug delivery pack housing 240 and the second part is a slot 315 (best seen in FIG. 6B) defined in the power pack housing 270. Inserting the tab 310 into the slot 315 (e.g., by bring the drug delivery pack 205 and power pack 210 together) unlocks the interlock 295. In turn, the cover 300 swings open from the close position exposing the trigger portion 275. The user can now press the trigger portion 275 to auto-inject a drug dose. Advantageously, in some examples of the wearable drug delivery device, the interlock 295 and/or the cover 300 are safety features.

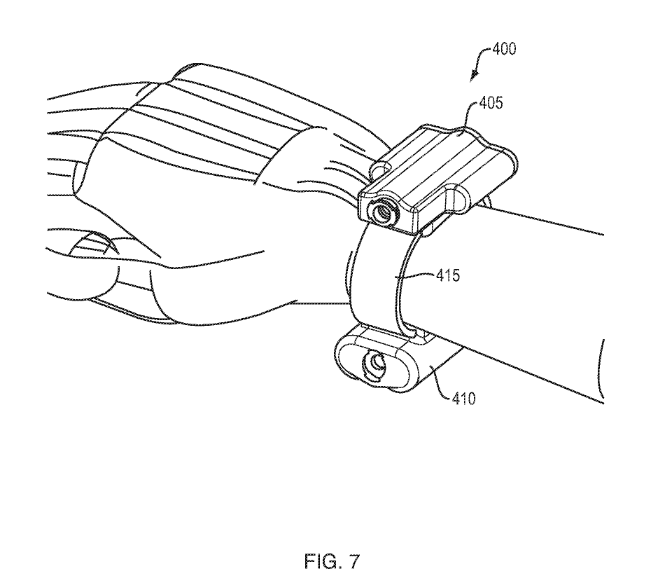

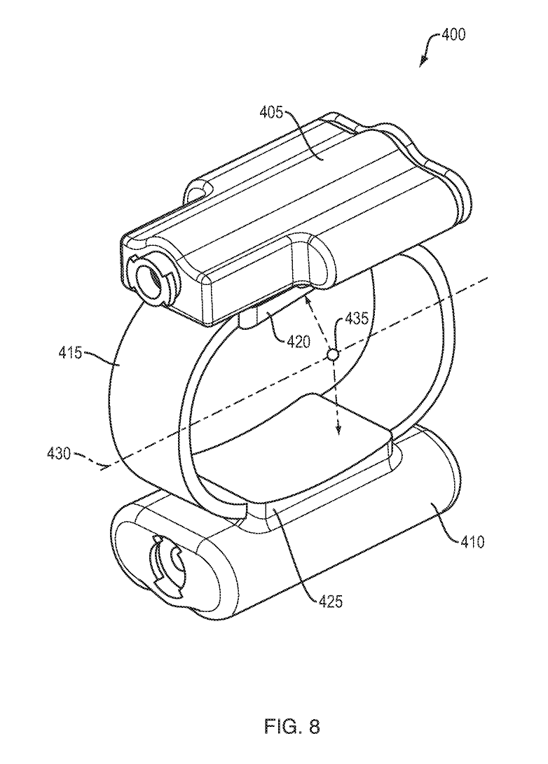

FIG. 7 shows yet another example wearable drug delivery device 400 being worn around a user's wrist. The wearable drug delivery device 400 includes a drug delivery pack 405, a power pack 410, and a band 415 for wrapping the drug delivery pack 405 and power pack 410 around the user's wrist. Other examples of the wearable drug delivery device 400 can be wrapped around the user's arm, leg or waist just to name a few possibilities. FIG. 8 shows the drug delivery pack 405 including a first concave surface 420 and the power pack 410 including a second concave surface 425 opposing the first concave surface 420 conforming to the round shape of the user's wrist, for example. FIG. 8 further shows a longitudinal axis 430 of the wearable drug delivery device 400. The first concave surface 420 and the second concave surface 425 extend in the direction of the longitudinal axis 430 and have concavity defined by a point 435 along the longitudinal axis 430.

To use the wearable drug delivery device 400 to auto-inject a drug dose, in one example, the user takes off the wearable drug delivery device 400 and removes the drug delivery pack 405 and power pack 410 from the band 415. The user then connects the drug delivery pack 405 to the power pack 410, as will be described next.

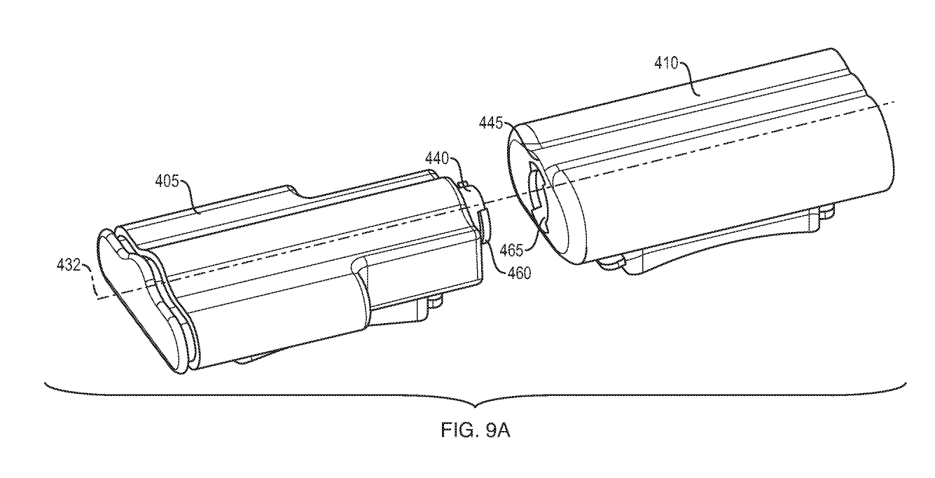

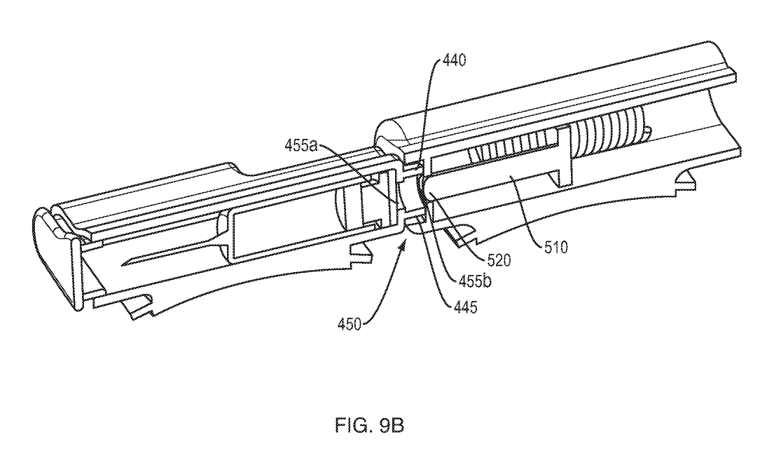

FIG. 9A shows the drug delivery pack 405 and the power pack 410 aligned with a longitudinal axis 432 of the wearable drug delivery device 400, as it would be assembled. The drug delivery pack 405 includes a protrusion 440 and the power pack 410 includes a recess 445 corresponding to the protrusion 440. As shown in FIG. 9B, inserting the protrusion 440 into the recess 445 forms as a rigid connection 450 between the drug delivery pack 405 and the power pack 410. There are openings 455a and 455b at both ends of the rigid connection 450 to provide a passageway for conveying energy from the power pack 410 to the drug delivery pack 405, as will be described later in greater detail.

Returning to FIG. 9A, in the example shown, the protrusion 440 and recess 445 include tabs 460 and corresponding slots 465. Twisting the drug delivery pack 405 and the power pack 410 together, so that the tabs 460 are received in the corresponding slots 465 locks the rigid connection 450. In some examples, the drug delivery pack protrusion 440 and power pack recess 445 are each coaxially aligned with the longitudinal axis 432. In other examples, the arrangement of protrusion and recess is reversed with the drug delivery pack 405 having the recess 445 and the power pack 410 having the protrusion 440.

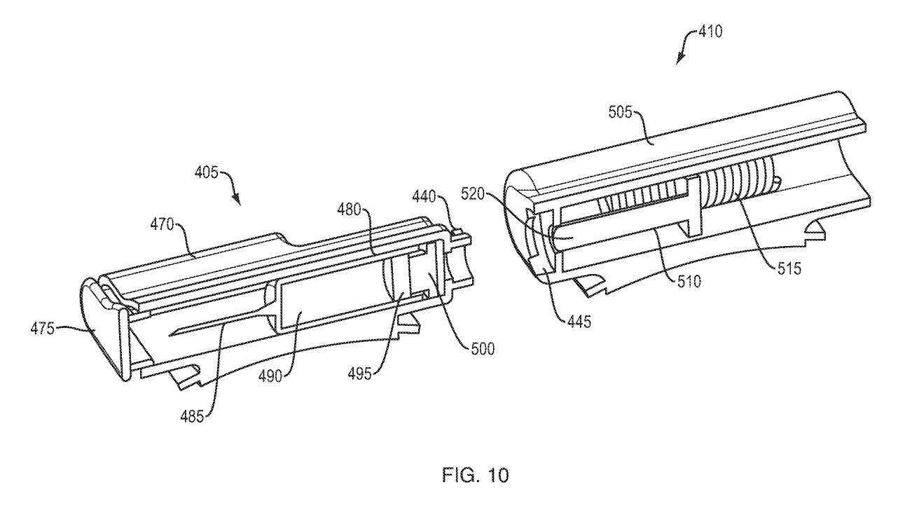

FIG. 10 shows an example of the drug delivery pack 405 having a housing 470 and a trigger portion 475. The housing 470 contains a syringe 480 consisting of a needle 485 at the forward end attached to a drug container 490 that is fitted with a sliding plunger 495 at the rear end. By way of non-limiting example, the drug container 490 can be filled with a dose of epinephrine or insulin. Moving the plunger 495 from a proximal position (shown) within the housing 470 to a distal position forces a drug dose out of the drug container 490. An actuator 500 moves the syringe 480 within the housing 470 in the direction of the longitudinal axis 432 (shown in FIG. 9A) from a proximal position (shown) to a distal position. For ease of reference, the proximal position within the housing 470 is called the “withdrawn position” and the distal position within the housing 470 is called the “extended position.” Additionally, the proximal-to-distal direction is referred to as the “downward direction,” and the opposite direction is the “upward direction.”

In a convenient example of the wearable drug delivery device, the drug delivery pack 405 has an open end opposite the protrusion 440. The open end is covered by a seal that keeps the needle 485 sterile when the syringe is in the withdrawn position. When the syringe is in the extended position, the needle 485 pierces through the seal and extends through the open end.

In the example shown, the power pack 410 further includes a housing 505 containing a driving pin 510 attached to a pre-compressed spring 515. The driving pin 510 is movable in the direction of the longitudinal axis 432 (shown in FIG. 9A) from a proximal position (shown) within the housing 505 to a distal position. As shown in FIG. 9B, when in the proximal position, a leading end 520 of the driving pin 510 sits within the opening 455b. Returning to FIG. 10, the pre-compressed spring 515 stores mechanical energy used to move the syringe 480 downwardly, to the insert the needle 485 into the user, and to inject the drug dose. A release mechanism (not shown) engages the pre-compressed spring 515 keeping it under compression. The release mechanism is coupled to the trigger portion 475 by a linkage or other similar type of connection. Moving the trigger portion 475 towards the proximal end of the wearable drug delivery device 400 activates the release mechanism. The release mechanism, in turn, disengages from the pre-compressed spring 515 thereby releasing the stored mechanical energy.

The release of energy causes the driving pin 510 to move to the distal position within the power pack housing 505 with the leading end 520 extending through the rigid connection 450. Referring in combination with FIG. 9B, the leading end portion 520 enters the drug delivery pack housing 470 through the opening 455a and pushes the actuator 500 in the downward direction (i.e., from proximal to distal). The actuator 500, in turn, moves the syringe 480 in the downward direction causing the needle 485 to exit the distal end of the wearable drug delivery device 400. The syringe 480 continues to move until a stop within the drug delivery pack housing 470 prevents further movement. At this point, the needle 485 extends a certain distance beyond the distal end of the wearable drug delivery device 400. Additional downward movement by the driving pin 510 causes the plunger 495 to move downwardly within the drug container 490, thereby expelling the drug dose from the drug container, through the needle 495, and into the user.

In other examples of the wearable drug delivery device 400, the pre-compressed spring 515 is replaced with a container of compressed gas or other similar stored energy source. The principles previously described above apply to such examples.

The invention may be embodied in other specific forms without departing from the spirit or essential characteristics thereof. The foregoing examples are therefore to be considered in all respects illustrative rather than limiting of the invention described herein. Also, the words comprise, include, and/or plural forms of each are open ended and include the listed parts and can include additional parts or steps that are not listed, and the term and/or is open ended and includes one or more of the listed parts or steps and combinations of the listed parts steps.

What is claimed is:

1. A wearable drug delivery device comprising:
a first housing comprising a stored energy source disposed within the first housing;
a second housing comprising a syringe movable within the second housing between a withdrawn position and an extended position, the syringe including a needle and a drug container in fluid communication with each other;
a flexible conductor connected between the first housing and the second housing, the flexible conductor configured to convey energy released from the stored energy source which moves the syringe from the withdrawn position to the extended position and extends the needle extending beyond the second housing.

2. The wearable drug delivery device of claim 1 wherein the stored energy source is a spring and wherein the flexible conductor is a Bowden cable.

3. The wearable drug delivery device of claim 1 wherein the stored energy source is a compressed gas and wherein the flexible conductor is a lumen.

4. The wearable drug delivery device of claim 1 further comprising:
a release mechanism coupled to the stored energy source and configured to discharge stored energy; and
a trigger mechanism disposed at one end of the second housing and coupled to the release mechanism such that in response to the trigger mechanism being activated, the release mechanism discharges the stored energy.

5. The wearable drug delivery device of claim 1 further comprising:
a release mechanism coupled to the stored energy source and configured to release stored energy; and
a trigger mechanism disposed at one end of the first housing and coupled to the release mechanism such that in response to the trigger mechanism being activated, the release mechanism discharges the stored energy.

6. The wearable drug delivery device of claim 1 wherein the first housing further includes a first concave surface and the second housing includes a second concave surface opposite the first concave surface, each of the first and second concave surfaces extends along a longitudinal axis lying between the first housing and the second housing and has a concavity defined by a point along the longitudinal axis.

7. The wearable drug delivery device of claim 6 wherein the first and second concave surfaces are configured to conform to the human wrist.

8. The wearable drug delivery device of claim 1 further comprising:
a cover hingedly attached to the first housing and operable between an initial close position and final open position; and
an interlock in releaseable engagement with the cover such that when unlocked the cover swings from the initial close position to the final open position.

9. The wearable drug delivery device of claim 8 wherein the interlock includes a recess defined by the first housing and a projection extending from the second housing, the interlock unlocks when the projection is inserted into the recess.

10. The wearable drug delivery device of claim 1 wherein the drug container is filled with any one of epinephrine and insulin.

11. The wearable drug delivery device of claim 1 wherein the second housing includes an open end through which the needle extends when the syringe is in the extended position; and
the wearable drug delivery device further comprising a seal at the open end for keeping the needle sterile when the syringe is in the withdrawn position.

12. A wearable drug delivery device comprising:
a first housing including a first longitudinal axis and a first concave surface extending in the direction of the first longitudinal axis, the concavity of the first concave surface defined by a point along an axis offset and parallel to the first longitudinal axis, the first housing further including a stored energy source disposed within the first housing;
a first connector disposed at an end of the first housing;
a second housing including a second longitudinal axis and a second concave surface extending in the direction of the second longitudinal axis, the concavity of the second concave surface defined by a second point along an axis offset and parallel to the second longitudinal axis;
a syringe movable within the second housing between a withdrawn position and an extended position, the syringe including a needle and a drug container in fluid communication with each other;
a second connector disposed at an end of the second housing, the second connector adapted to couple the first connector of the first housing; and
a band releasably coupled to the first housing and the second housing, such that when coupled to the band, the first concave surface of the first housing is opposite the second concave surface of the second housing.

13. The wearable drug delivery device of claim 12 wherein the stored energy source is any one of pre-compressed spring or compressed gas.

14. The wearable drug delivery device of claim 12 wherein the first connector and the second connector are coaxially aligned with the first longitudinal axis and the second longitudinal axis, respectively.

15. The wearable drug delivery device of claim 12 wherein the first connector is a recess defined in the end of the first housing and the second connector is a protrusion extending from the end of the second housing.

16. The wearable drug delivery device of claim 15 wherein the recess includes radial slots and the protrusion includes radial tabs corresponding to the radial slots.

17. The wearable drug delivery device of claim 12 wherein the first connector and the second connector, when connected, form a passageway between the first and second housings through which a driving pin slides and moves the syringe from the withdrawn position to the extended position; and wherein the needle extends beyond the second housing when the syringe is in the extended position.

18. The wearable drug delivery device of claim 12 further comprising a trigger portion disposed at an end of the second housing opposite the second connector, the trigger portion when activated discharges stored energy, which, in turn, moves the syringe from the withdrawn position to the extended position; and wherein the needle extends beyond the second housing when the syringe is in the extended position.

19. The wearable drug delivery device of claim 12 wherein the drug container is filled with any one of epinephrine and insulin.

20. The wearable drug delivery device of claim 12 wherein the second housing includes an open end opposite the second connector through which the needle extends when the syringe is in the extended position; and
the wearable drug delivery device further comprising a seal at the open end for keeping the needle sterile when the syringe is in the withdrawn position.
